(19) United States
(12) Patent Application Publication
(10) Pub. No.: US 2015/0012059 A1
(43) Pub. Date: Jan. 8, 2015

(54) DEVICE FOR COCHLEAR IMPLANT WITH SENSOR AND ELECTRODE

(71) Applicant: SNU R&DB FOUNDATION, Seoul (KR)

(72) Inventors: Wan-Doo Kim, Daejeon (KR); Shin Hur, Daejeon (KR); Seung Ha Oh, Seoul (KR); Juyong Chung, Yongin-si (KR)

(21) Appl. No.: 14/376,141

(22) PCT Filed: Feb. 4, 2013
(86) PCT No.: PCT/KR2013/000885

§ 371 Date: Aug. 1, 2014

(30) Foreign Application Priority Data
Feb. 2, 2012 (KR) 10-2012-0010989

(51) Int. Cl. A61N 1/36

(52) U.S. Cl. CPC A61N 1/36032
USPC 607/57

(57) ABSTRACT

Disclosed is a device for a cochlear implant with a sensor and an electrode to sense vibration of a sound and stimulate the auditory nerve, the device comprising a sensor unit inserted in a scala tympani of the cochlea, sensing vibration caused by a sound from the cochlea, converting the sensed vibration into an electrical signal, and transmitting the converted electrical signal to the outside of the cochlea.

### TECHNICAL FIELD

The present invention relates to a device for a cochlear implant with a sensor and an electrode, and more particularly, to a device for a cochlear implant with a sensor and an electrode that is capable of receiving vibration caused by a sound from a basilar membrane of a cochlea through a tube-type sensor inserted in a scala tympani of the cochlea.

### BACKGROUND ART

A sound is transferred to a cochlea through an eardrum and auditory ossicles. Vibration caused by the sound transferred to the cochlea is transferred to a hair cell through vibration of a basilar membrane, and the hair cell converts a physical signal into an electrical signal. The electrical signal stimulates the auditory nerve to allow the brain to recognize the sound. Thus, when the cochlea is severely damaged, the profound deaf (i.e., patients who are extremely hard of hearing) may suffer from hearing loss to a point that they cannot have an everyday conversation even with a hearing aid. Thus, in the recent technology, a cochlear implant system has been developed to interpret an audio signal delivered from the outside and an electrical signal is directly transferred to the auditory nerve to solve hearing loss.

Here, the related art cochlear implant may be divided into an external part and an internal part. In other words, the cochlear implant system is configured to include an external device for receiving a sound from the outside of a human body, and an internal device insertedly positioned in a human body and stimulating the auditory nerve. The external part installed outside of a human body includes a microphone (or a sender), a speech processor (or a speech synthesizer), and a transmission antenna (or a transmitter), and a combination of the microphone and the transmission antenna is called a headset. The internal part implanted in a body includes a receptor/stimulator (or a receiver) and an electrode.

Thus, in the related art cochlear implant system, an audio signal transferred from the microphone attached to the outside of a human body is amplified and filtered by an external speech processor, and is converted into an electrical signal and transferred to spiral ganglion through the electrode implanted in the cochlea. To this end, the related art cochlear implant system requires a sender for receiving a sound from the outside of a living body and an external device for analyzing and processing the audio signal and converting the same into an electrical signal, which consumes much power.

In this process, the related art cochlear implant system omits a sound amplifying process of the eardrum or the auditory ossicles or a frequency discrimination mechanism of a basilar membrane. Thus, efforts to generate a low power device frequently using a sound transmission mechanism of the external ear and the middle ear by developing a conversion device that may replace a function of a hair cell of a human body that converts mechanical vibration into an electrical signal are required.

Also, in the case of the related art cochlear implant system, the separate equipment is required to be constantly attached to the outside of a human body, causing problems in that it takes a long time for a user to become accustomed thereto as part of his body and a third party may recognize it.

### PRIOR ART DOCUMENT

### Patent Document

Korean Patent Registration No. 10-0932204

The prior art relates to a frequency analyzer of a cochlear implant having a MEMS structure with a self-power supply function, wherein: an upper structure is stacked on a lower structure; the upper structure includes a first substrate and a nano-wire contact portion formed on a lower portion of the first substrate, the lower portion having a sawtooth shape and coated with platinum; the lower structure includes a second substrate having a certain space (space portion) formed therein to hold a fluid therein and having a structure in which an upper portion is open; a basilar membrane is formed on the fluid present in the space portion of the second substrate; a plurality of first electrodes are continuously formed on the basilar membrane such that intervals between the first intervals and widths (w) and lengths (L) of the first electrodes are different; and a nano-wire is grown on an upper portion of the first electrodes in a certain direction and having piezoelectric and semiconductor characteristics. It further includes a sound wave entrance as a passage in which a sound wave makes a fluid in the space portion of the lower structure flow when the sound wave is generated, the nano-wire comes into contact with a nano-wire contact portion of the upper structure when the upper structure is stacked on the lower structure, and when a sound wave is generated, the sound wave moves the fluid of the lower structure, a particular position of the basilar membrane is moved by a particular frequency component of the sound wave, the nano-wire in contact with the first electrode on an upper portion of the basilar membrane is deformed, and the deformed nano-wire comes into contact with a corresponding nano-wire contact portion of the upper structure, and thus a nano-wire fixed to a shorter first electrode and a nano-wire fixed to a longer first electrode generate electrical signals (currents) having a particular frequency corresponding to a high frequency component and a low frequency component, respectively.

The above information disclosed in this Background section is only for enhancement of understanding of the background of the invention and therefore it may contain information that does not form the prior art that is already known in this country to a person of ordinary skill in the art.

### DISCLOSURE OF INVENTION

### Technical Problem

Thus, a recent cochlear implant system requires a technique of inserting an external device, which is exposed to the outer side of a human body, into the inner side of the human body.

### Solution to Problem

The present invention has been made in an effort to provide a device for a cochlear implant with a sensor and an electrode having advantages of eliminating the necessity of installation of a microphone at the outside of a human body, by receiving vibration of a sound from a basilar membrane of the cochlea.

The present invention has also been made in an effort to use a frequency analyzer of a cochlear implant having a MEMS structure having a self-power supply function, as a sensor, in order to generate a low power device using an in-vivo mechanism.

The present invention has also been made in an effort to provide an environment in which an operation may be easily performed by inserting a tube-type sensor for receiving vibration of a sound together with an electrode into the cochlea.

The present invention has also been made in an effort to effectively insert a tube into an annular cochlea by forming the tube with a flexible material.

The present invention has also been made in an effort to provide an environment in which vibration according to a sound can be easily sensed by using various types of sensors.

An exemplary embodiment of the present invention provides a device for a cochlear implant with a sensor and an electrode to sense vibration of a sound and stimulate the auditory nerve. The sensor unit is inserted in a scala tympani of the cochlea, sensing vibration caused by a sound from the basilar membrane of the cochlea, converting the sensed vibration into an electrical signal, and transmitting the converted electrical signal to the outside of the cochlea. The electrode unit receiving processed signal from amplifier or speech processor is also inserted in a scala tympani of the cochlea and stimulates the auditory nerve.

Another embodiment of the present invention provides a device for a cochlea implant to sense vibration of a sound to perform transmission and reception with an amplifier or a speech processor, and simulate an auditory nerve, including: a tube inserted into a scala tympani such that one surface thereof is located near a basilar membrane of a cochlea; a sensor unit installed in the tube, sensing vibration according to a sound from the cochlea, converting the sensed vibration into an electrical signal, and transmitting the converted electrical signal to the amplifier or the speech processor; and a plurality of electrode units installed in the tube and stimulating the auditory nerve upon receiving a processed signal from the amplifier or the speech processor.

Yet another embodiment of the present invention provides a device for a cochlea implant to sense vibration of a sound and perform transmission and reception with a speech processor, and simulate an auditory nerve, including: a tube inserted into a scala tympani such that one surface thereof is located near a basilar membrane of a cochlea; a sensor unit installed in the tube, sensing vibration caused by a sound from the basilar membrane of the cochlea, and converting the sensed vibration into an electrical signal; an amplifying unit installed in the tube, receiving the electrical signal from the sensor unit, amplifying the received electrical signal, and transmitting the amplified signal to the speech processor; and a plurality of electrode units installed in the tube and stimulating the auditory nerve upon receiving a processed signal from the speech processor.

The tube may have a sectional area that is gradually reduced from one end to the other end, the one end being disposed in an inlet portion of the cochlea and the other end being disposed in an end portion corresponding to the inlet portion of the cochlea, one surface of the tube, as a parallel section, may be parallel to the basilar membrane and the other surface of the tube may have a circular shape, the sensor unit may be positioned on the one surface of the tube, and the electrode unit may be positioned on the other surface of the tube.

The sensor unit and the electrode unit may be in contact with each other within the tube, and are paired.

The tube may be made of a flexible material, and the material may be a polymer.

The sensor unit may include a nano-pillar, a piezoelectric element, or a photosensor.

### Advantageous Effects of Invention

As described above, the device for a cochlear implant with a sensor and an electrode according to an embodiment of the present invention receives vibration of a sound from the basilar membrane of the cochlea, thus providing a cochlear implant without the necessity of installation of a microphone at the outside of a body.

In the device for a cochlear implant with a sensor and an electrode according to an embodiment of the present invention, a frequency analyzer of a cochlear implant having a MEMS structure with a self-power supply function is used as a sensor in order to generate a low power device using an in-vivo mechanism.

In the device for a cochlear implant with a sensor and an electrode according to an embodiment of the present invention, since the sensor for receiving vibration of a sound together with an electrode is inserted into the scala tympani, an environment in which an operation is easily performed can be provided.

In the device for a cochlear implant with a sensor and an electrode according to an embodiment of the present invention, since the tube inserted into the cochlea is made of a flexible material, the tube can be effectively inserted into the annular cochlea.

Another object of the present invention provides an environment in which vibration according to a sound can be easily sensed by using various types of sensors.

### BRIEF DESCRIPTION OF DRAWINGS

FIG. 1 is a partial perspective view illustrating a section of a device for a cochlear implant with a sensor and an electrode and a sectional view thereof according to an embodiment of the present invention.

FIG. 2 is a side view of the device for a cochlear implant with a sensor and an electrode of FIG. 1.

FIG. 3 is a side view of the device for a cochlear implant with a sensor and an electrode according to another embodiment of the present invention.

FIG. 4 is a sectional view illustrating the device for a cochlear implant with a sensor and an electrode of FIG. 1 inserted in the scala tympani of the cochlea.

FIG. 5 is a sectional view illustrating a sensor unit implemented as a nano-pillar in the device for a cochlear implant with a sensor and an electrode of FIG. 4.

FIG. 6 is a sectional view of a device for a cochlear implant with a sensor and an electrode according to a second embodiment of the present invention.

FIG. 7 is a flowchart illustrating a process of processing a signal according to the device for a cochlear implant with a sensor and an electrode according to an embodiment of the present invention.

FIG. 8 is a perspective view of the device for a cochlear implant with a sensor and an electrode according to an embodiment of the present invention.

FIG. 9 is a view illustrating an overall system of the cochlear implant installed according to an embodiment of the present invention.

### MODE FOR THE INVENTION

Embodiments of the present invention will now be described in detail with reference to the accompanying drawings. The invention may, however, be embodied in many different forms and should not be construed as being limited to the embodiments set forth herein. The present embodiments are provided so that this disclosure will be thorough and complete, and will fully convey the scope of the invention to those skilled in the art. In the drawings, the shapes and dimensions may be exaggerated for clarity.

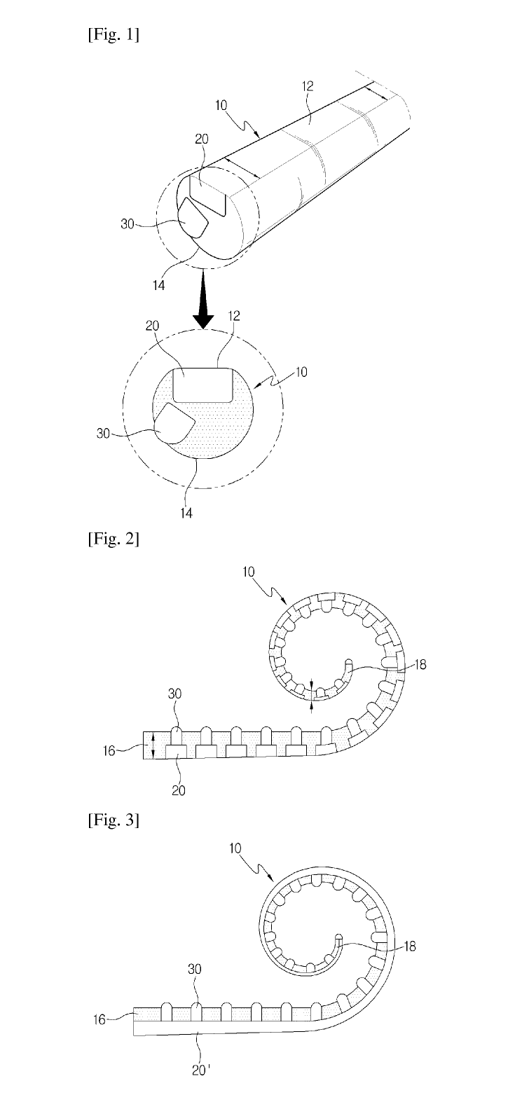

Hereinafter, embodiments of the present invention will be described in detail with reference to FIGS. 1 to 9.

FIG. 1 is a partial perspective view illustrating a section of a device for a cochlear implant with a sensor and an electrode and a sectional view thereof according to an embodiment of the present invention.

The device for a cochlear implant with a sensor and an electrode according to an embodiment of the present invention is formed as a tube 10 and includes a sensor unit 20 and an electrode unit 30 installed in the tube 10.

The tube 10 is inserted in a ring shape along a scala tympani of the cochlea, and includes the sensor unit 20 for directly sensing vibration by a sound in the cochlea and the electrode unit 30 for transferring electrical stimulation to the auditory nerve in the cochlea. That is, the device for a cochlear implant with a sensor and an electrode according to an embodiment of the present invention is the annular tube 10 inserted into the cochlea, it directly senses vibration caused by a sound within the tube 10, and simulates the auditory nerve through an electrode installed in the tube 10.

The tube 10 is formed to be easily inserted into the annular cochlea and easily sense vibration from a basilar membrane of the cochlea. Thus, according to an embodiment of the present invention, the tube 10 is inserted as a ring shape through the scala tympani of the cochlea, and is inserted such that one surface 12 of the tube 10 inserted along the scala tympani is located near the basilar membrane of the cochlea.

Also, according to an embodiment of the present invention, the tube 10 is configured to have a sectional area that is gradually reduced from one end 16 to the other end 18. The tube 10 is disposed such that one end 16 of the tube 10 is disposed in a portion (i.e., an inlet portion of the scala tympani) introduced into the cochlea and the other end 18 of the tube 10 is disposed in an end portion (i.e., an end portion corresponding to the inlet portion of the scala tympani) within the cochlea.

That is, the tube 10 is introduced from the other end 18 thereof along the scala tympani of the annular cochlea and the other end 18 is disposed in the central portion of the annular cochlea. Thus, the device for a cochlear implant with a sensor and an electrode according to an embodiment of the present invention can be tightly attached to an inner wall of the cochlea along the scala tympani of the cochlea whose sectional area is gradually reduced in a ring shape.

One surface 12 of the tube 10 is a section that is parallel to the basilar membrane and another surface 14 has a circular shape, by which the tube 10 is formed to be tightly attached to an inner wall of the cochlea along the scala tympani in the cochlea. Thus, since the one surface 12 of the tube 10 is disposed to be parallel to the basilar membrane of the cochlea, the tube 10 can easily receive vibration from the basilar membrane. Also, since the other surface 14 of the tube is positioned at the inner wall side of the cochlea, the tube 10 is formed to easily stimulate the auditory nerve through the electrode unit 30 as described hereinafter.

Also, according to an embodiment of the present invention, the tube 10 is made of a flexible material, and the flexible material may be a polymer. Thus, the tube 10 has an advantage in that it can be easily inserted into the annular cochlea because of the flexible material.

The sensor unit 20 is installed in the tube 10, is disposed to be located near the basilar membrane, and senses vibration caused by a sound from the basilar membrane. The sensor unit 20 converts the sensed vibration into an electrical signal. That is, the sensor unit 20 serves to convert a physical signal based on vibration into an electrical signal. Also, the sensor unit 20 transmits the electrical signal to an amplifier or a speech processor installed outside the tube 10.

Thus, the sensor unit 20 is disposed on the parallel surface 12 of the tube 10 and is positioned to be parallel to the basilar membrane, thus easily sensing vibration from the basilar membrane.

Also, the sensor unit 20 according to an embodiment of the present invention is configured as a nano-pillar, a piezoelectric element, or a photosensor. Also, a size of the sensor unit 20 may be within a few micrometers.

That is, in the sensor unit 20, various types of sensors may be attached according to circumstances to effectively sense vibration.

The electrode unit 30 is installed in the tube 10, receives a processed signal from the outside of the tube 10, and transfers stimulation to the auditory nerve within the cochlea.

Thus, the electrode unit 30 stimulates the auditory nerve upon receiving the processed signal from the amplifier or the speech processor, which includes a plurality of electrodes within the tube 10.

Here, the electrode unit 30 is disposed on the other surface 14 of the tube 10, and is positioned to be tightly attached to the auditory nerve through the other surface 14 having a circular shape, thus easily transferring stimulation to the auditory nerve.

FIG. 2 is a side view of the device for a cochlear implant with a sensor and an electrode of FIG. 1.

The device for a cochlear implant with a sensor and an electrode according to an embodiment of the present invention illustrated in FIG. 2 includes a plurality of electrode units 30 and a plurality of sensor units 20 within the tube 10. It is illustrated that the respective electrode units 30 and the respective sensor units 20 may be independent without being in contact with each other, but they may be connected as pairs within the tube 10. Thus, the respective electrode units 30 and the sensor units 20 are in a mutually independent relationship.

The tube 10 may have an annular shape and may be inserted along the scala tympani of the cochlea. Also, the tube 10 is formed such that a sectional area thereof is reduced from one end 16 to the other end 18, so as to be easily inserted into the scala tympani having a sectional size whose size is gradually reduced, and located near the basilar membrane of the cochlea and an inner wall of the cochlea.

The sensor units 20 and the electrode units 30 installed in the tube 10 process a high frequency signal to a low frequency signal from one end 16 to the other end 18 of the tube 10. That is, one end 16 of the tube 10 processes a high frequency signal, and the other end 18 of the tube 10 processes a low frequency signal.

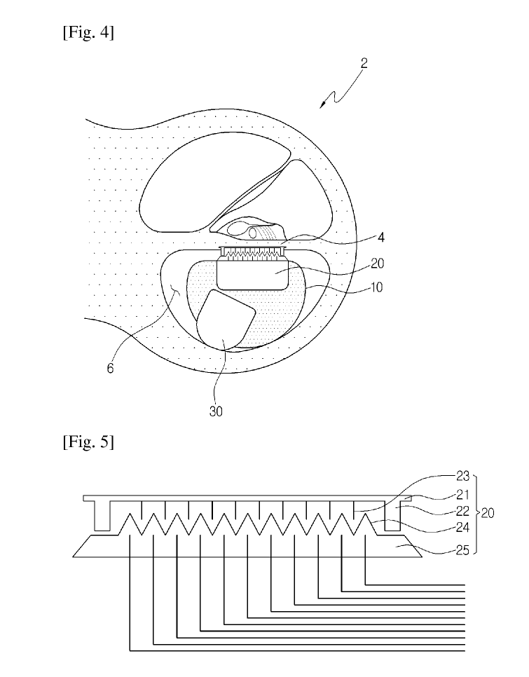

FIG. 3 is a side view of the device for a cochlear implant with a sensor and an electrode according to another embodiment of the present invention.

FIG. 3 illustrates that the electrode units 30 are continuously formed on one surface 12 of the tube 10. Thus, a sensor unit 20′ is continuously connected to the basilar membrane in the cochlea. Through this structure, the sensor unit 20′ has an advantage in that it can continuously and effectively sense vibration from the basilar membrane.

FIG. 4 is a sectional view illustrating the device for a cochlear implant with a sensor and an electrode of FIG. 1 inserted in the cochlea.

As illustrated in FIG. 4, the tube 10 is inserted along the scala tympani 6 illustrated in the section 2 of the cochlea. The tube 10 includes the sensor unit 20 and the electrode unit 30, and the sensor unit 20 and the electrode unit 30 are located near the basilar membrane 4 of the cochlea and an inner wall of the cochlea, respectively. Thus, the sensor unit 20 is positioned to be parallel to the basilar membrane 4 to sense vibration caused by a sound from the basilar membrane 4, and the electrode 30 adjacent to the inner wall of the cochlea stimulates the auditory nerve of the cochlea.

FIG. 5 is a sectional view illustrating a sensor unit implemented as a nano-pillar in the device for a cochlear implant with a sensor and an electrode of FIG. 4.

The sensor unit 20 illustrated in FIG. 5 is configured as a nano-pillar type of MEMS structure. A first substrate 21 is formed as a flat plate so as to be disposed to be parallel to the basilar membrane 4, and includes a plurality of nano-wires 23 sensing vibration from the basilar membrane 4. Also, in the sensor unit 20, the first substrate 21 and a second substrate 25 are connected by a support 22 of the first substrate 21 and disposed to be parallel to each other. The second substrate 25 senses vibration of the nano-wires 23 by vibration through a plurality of protrusions 24, and transmits the sensed signal to the outside of the tube 10 through an electric wire. The nano-pillar type of MEMS structure is a frequency analyzer of a cochlear implant having a self-power supply function, which is implemented as a low power device using an in-vivo mechanism.

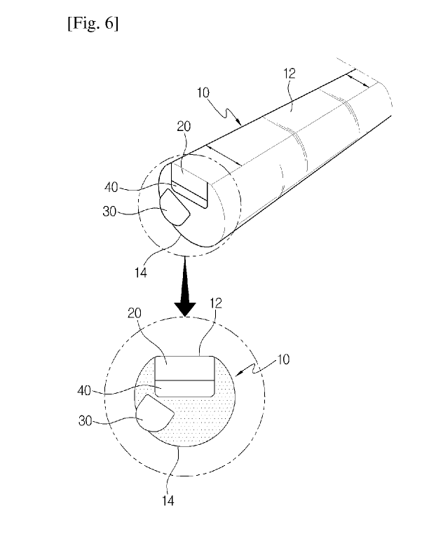

FIG. 6 is a sectional view of a device for a cochlear implant with a sensor and an electrode according to a second embodiment of the present invention.

The device for a cochlear implant with a sensor and an electrode illustrated in FIG. 6 includes an amplifying unit 40 connected to the sensor unit 20 within the tube 10, in addition to the sensor unit 20 and the electrode unit 30.

Thus, the amplifying unit 40 amplifies vibration sensed from the sensor unit 20 and transmits the amplified signal to the outside of the tube 10. The vibration signal sensed by the sensor unit 20 may have weak strength, so noise may be generated while the vibration signal is transmitted to the outside of the tube 10. That is, the vibration signal may be vulnerable to noise. However, since the amplifying unit 40 illustrated in FIG. 6 amplifies the signal from the sensor unit 20 and transmits the same, it is resistant to noise.

Hereinafter, a process of transmitting and receiving vibration based on a sound through the device for a cochlear implant with a sensor and an electrode according to an embodiment of the present invention will be described.

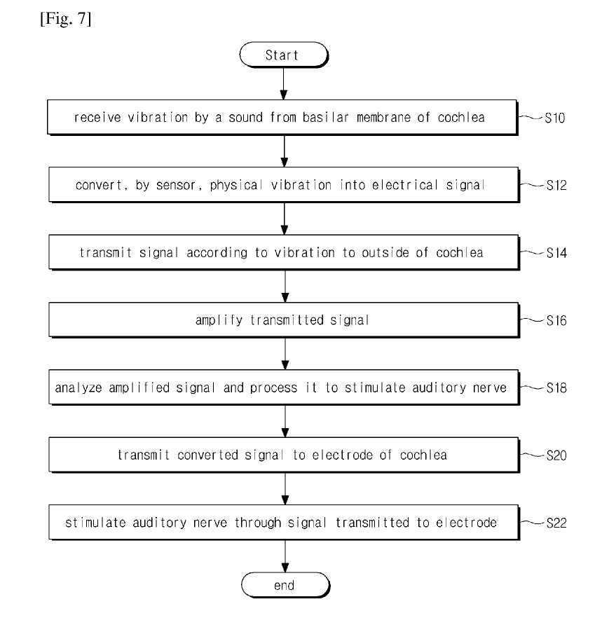

FIG. 7 is a flowchart illustrating a process of processing a signal according to the device for a cochlear implant with a sensor and an electrode according to an embodiment of the present invention.

The sensor unit 20 senses vibration caused by a sound from the basilar membrane of the cochlea, and converts the sensed vibration into an electrical signal (S10, S12). The sensor unit 20 transmits the electrical signal described by frequency band through a plurality of sensors to the outside of the cochlea (S14). Thus, a sound is transferred to the basilar membrane of the cochlea through the eardrum and the auditory ossicles, and the sensor unit 20 within the cochlea directly senses vibration of the basilar membrane.

The amplifying unit 40 installed outside of the cochlea amplifies the signal transmitted from the sensor unit 20, and the speech processor analyzes the amplified signal from the amplifying unit 40 and processes it to stimulate the auditory nerve (S16, S18). Of course, according to an embodiment of the present invention, the amplifying unit 40 may be installed in the tube 10 and transmit the amplified signal to the outside of the cochlea.

Also, a plurality of electrode units 30 receive the converted signal from the outside of the cochlea and stimulate the auditory nerve, respectively (S12, S22).

Thus, the method for processing a signal through the cochlear implant with a sensor and an electrode according to an embodiment of the present invention includes a sound sensing step, a conversion step, a sound amplifying step, a processing step, and an auditory nerve stimulating step.

The sound sensing step is a step of sensing vibration by a sound from the basilar membrane 4 of the cochlea.

The converting step is a step of converting the sensed vibration into an electrical signal by the sensor unit 20.

The sound amplifying step is a step of receiving the electrical signal and amplifying the electrical signal by the amplifying unit.

The processing step is a step of receiving the amplified signal and processing the amplified signal by the speech processor.

The auditory nerve stimulating step is a step of receiving the processed signal and stimulating the auditory nerve through the processed signal by the electrode unit 30.

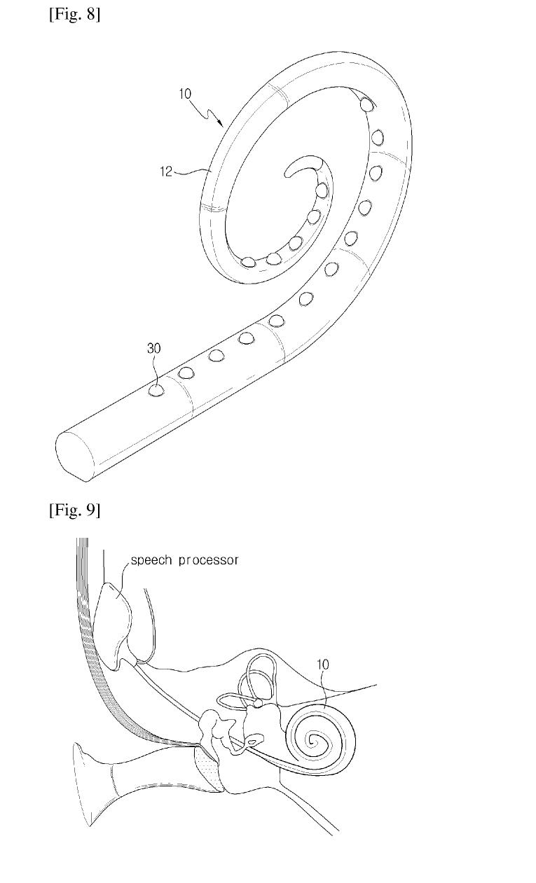

FIG. 8 is a perspective view of the device for a cochlear implant with a sensor and an electrode according to an embodiment of the present invention, and FIG. 9 is a view illustrating an overall system of the cochlear implant installed according to an embodiment of the present invention.

The device for a cochlear implant with a sensor and an electrode illustrated in FIG. 8 is a tube 10 having a shape of a cochlea. A sectional area of the tube 10 is gradually reduced from one end 16 toward the other end 18, and a plurality of electrode units 30 are disposed on the other circular surface 14. Although not shown, a plurality of sensor units 20 are mounted on one parallel surface 12.

As illustrated in FIG. 9, the device for a cochlear implant with a sensor and an electrode is insertedly installed in a ring shape within the cochlea and connected to a speech processor, or the like, installed outside the cochlea. Thus, according to an embodiment of the present invention, a complete-implantation type of cochlear implant system in which all the devices of the cochlear implant are disposed in the interior of a human body can be implemented. Also, although not shown, a signal is mapped and power is charged through a coil connected to the speech processor.

Thus, in the device for a cochlear implant with a sensor and an electrode according to an embodiment of the present invention, instead of a microphone installed outside of a human body and receiving a sound, the tube 10 including the sensor unit 20 is installed in the scala tympani of the cochlea to directly receive a sound from the basilar membrane of the cochlea. The sensor unit 20 converts sensed vibration into an electrical signal by a nano-pillar or a piezoelectric element, and transmits the converted electrical signal to the outside of the cochlea so as to be processed into a signal that can be recognized by a human body.

Here, the speech processor processes the electrical signal transmitted from the cochlea such that it can be recognized in a body, and transfers the processed signal to the electrode insertedly positioned in the cochlea. The electrode stimulates the auditory nerve through the electrical signal transferred from the speech processor.

Thus, since the device for a cochlear implant with a sensor and an electrode according to an embodiment of the present invention receives vibration of a sound from the basilar membrane of the cochlea, an artificial cochlea not requiring installation of a microphone at an outer side of a human body can be provided.

Also, in order to generate a low power device using an in-vivo mechanism, the device for a cochlear implant with a sensor and an electrode according to an embodiment of the present invention uses a frequency analyzer of an artificial cochlea having a MEMS structure with a self-power supply function, as a sensor.

Also, in the device for a cochlear implant with a sensor and an electrode according to an embodiment of the present invention, the sensor receiving vibration of a sound together with the electrode is insertedly positioned in the scala tympani, thus providing an environment in which an operation can be easily performed.

Also, in the device for a cochlear implant with a sensor and an electrode according to an embodiment of the present invention, the tube 10 inserted into the cochlea is made of a flexible material so as to be effectively inserted into the annular cochlea.

Also, another object of the present invention provides an environment in which vibration based on a sound is easily sensed by using various types of sensors.

While the configuration and operation of the device for a cochlear implant with a sensor and an electrode according to the present invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

### DESCRIPTION OF SYMBOLS

2: section of cochlea 4: basilar membrane

6: scala tympani 10: tube

12: one surface of tube 14: other surface of tube

16: one end of tube 18: other end of tube

20: sensor unit 21: first substrate

22: support 23: nano-wire

24: protrusion 25: second substrate

30: electrode unit 40: amplifying unit

1-16. (canceled)

17. A device for a cochlear implant with a sensor and an electrode to sense vibration of a sound and stimulate the auditory nerve, the device comprising
a sensor unit inserted in a scala tympani of the cochlea, sensing vibration caused by a sound from the cochlea, converting the sensed vibration into an electrical signal, and transmitting the converted electrical signal to the outside of the cochlea.

18. The device of claim 17, wherein the sensor unit senses vibration caused by a sound from the basilar membrane of the cochlea.

19. The device of claim 17, wherein the sensor unit is configured as one of a nano-pillar, piezoelectric element, and photosensor.

20. A device for a cochlea implant to sense vibration of a sound to perform transmission and reception with an amplifier or a speech processor, and simulate the auditory nerve, the device comprising:
a tube inserted into a scala tympani such that one surface thereof is located near a basilar membrane of a cochlea;
a sensor unit installed in the tube, sensing vibration according to a sound from the cochlea, converting the sensed vibration into an electrical signal, and transmitting the converted electrical signal to the amplifier or the speech processor; and
a plurality of electrode units installed in the tube and stimulating the auditory nerve upon receiving a processed signal from the amplifier or the speech processor.

21. The device of claim 20, wherein the sensor unit senses vibration caused by a sound from the basilar membrane of the cochlea.

22. The device of claim 20, wherein the tube has a sectional area that is gradually reduced from one end to the other end, the one end being disposed in an inlet portion of the cochlea and the other end being disposed in an end portion corresponding to the inlet portion of the cochlea.

23. The device of claim 20, wherein one surface of the tube, as a parallel section, is parallel to the basilar membrane, and the other surface of the tube has a circular shape.

24. The device of claim 23, wherein the sensor unit is positioned on the one surface of the tube.

25. The device of claim 23, wherein the electrode unit is positioned on the other surface of the tube.

26. The device of claim 20, wherein the sensor unit and the electrode unit are in contact with each other within the tube, and are paired.

27. The device of claim 20, wherein the tube is made of a flexible material.

28. The device of claim 27, wherein the material is a polymer.

29. The device of claim 20, wherein the sensor unit is configured as one of a nano-pillar, piezoelectric element, and photosensor.

30. A device for a cochlea implant to sense vibration of a sound to perform transmission and reception with a speech processor, and simulate an auditory nerve, the device comprising:
a tube inserted into a scala tympani such that one surface thereof is located near a basilar membrane of a cochlea;
a sensor unit installed in the tube, sensing vibration caused by a sound from the cochlea, and converting the sensed vibration into an electrical signal;
an amplifying unit installed in the tube, receiving the electrical signal from the sensor unit, amplifying the received electrical signal, and transmitting the amplified signal to the speech processor; and
a plurality of electrode units installed in the tube and stimulating the auditory nerve upon receiving a processed signal from the speech processor.

31. The device of claim 30, wherein the sensor unit senses vibration caused by a sound from the basilar membrane of the cochlea.

32. The device of claim 30, wherein the tube has a sectional area that is gradually reduced from one end to the other end, the one end being disposed in an inlet portion of the cochlea and the other end being disposed in an end portion corresponding to the inlet portion of the cochlea.

33. The device of claim 30, wherein one surface of the tube, as a parallel section, is parallel to the basilar membrane, and the other surface of the tube has a circular shape.

34. The device of claim 33, wherein the sensor unit is positioned on the one surface of the tube.

35. The device of claim 33, wherein the electrode unit is positioned on the other surface of the tube.

36. The device of claim 30, wherein the tube is made of a flexible material.

37. The device of claim 36, wherein the material is a polymer.

38. The device of claim 30, wherein the sensor unit is configured as one of a nano-pillar, piezoelectric element, and photosensor.

39. A method for processing a signal through a cochlear implant with a sensor and an electrode, the method comprising:
sensing, by a sensor unit, vibration caused by a sound from a basilar membrane of a cochlea;
converting, by the sensor unit, the sensed vibration into an electrical signal;
receiving, by an amplifying unit, the electrical signal and amplifying the electrical signal;
receiving, by a speech processor, the amplified signal and processing the amplified signal; and
receiving, by an electrode unit, the processed signal and stimulating an auditory nerve through the processed signal.

40. The method of claim 39, wherein, in the sensing of a sound, the inlet portion of the cochlea is tightly attached to the basilar membrane to sense the sound, and the end portion of the cochlea corresponding to the inlet portion is separated from the basilar membrane to sense the sound.
